(19) United States
(12) Patent Application Publication
(10) Pub. No.: US 2022/0064083 A1
(43) Pub. Date: Mar. 3, 2022

(54) INTEGRATED PROCESS FOR PRODUCING ACETYLENE

(71) Applicant: UOP LLC, Des Plaines, IL (US)

(72) Inventors: Gregory Funk, Carol Stream, IL (US); John Goodman, Katy, TX (US); Parag Jain, Morton Grove, IL (US)

(21) Appl. No.: 17/341,544

(22) Filed: Jun. 8, 2021

Related U.S. Application Data
(60) Provisional application No. 63/029,859, filed on May 26, 2020.

(51) Int. Cl. C07C 2/78; C07C 7/12; F23R 3/02; F23R 3/28

(52) U.S. Cl. CPC C07C 2/78; C07C 7/12; F23R 2900/03282; F23R 3/28; F23R 3/02

(57) ABSTRACT

An integrated process for producing acetylene is provided. The process comprises separating a gas stream comprising methane from a fuel gas stream in a fuel gas recovery unit of a process. A fuel and an oxidizer are combusted in a combustion zone of a pyrolytic reactor to create a combustion gas stream, wherein the pyrolytic reactor is integrated with the fuel gas recovery unit via the gas stream comprising methane. A light hydrocarbon stream comprising all or a first portion of the gas stream comprising methane is injected into a supersonic combustion gas stream to create a mixed stream. The velocity of the mixed stream is transitioned from supersonic to subsonic in a reaction zone of the pyrolytic reactor to produce a reaction mixture comprising acetylene, methane, carbon oxides, and hydrogen. The reaction mixture is separated to provide an acetylene stream.

This application claims priority from U.S. application 63/029,859 filed May 26, 2020.

The field relates to an integrated process for producing acetylene. More particularly, the field relates to an integrated pyrolysis process for producing acetylene.

### BACKGROUND

Light olefin materials, including ethylene and propylene, represent a large portion of the worldwide demand in the petrochemical industry. Light olefins are used in the production of numerous chemical products via polymerization, oligomerization, alkylation and other well-known chemical reactions. These light olefins are essential building blocks for the modern petrochemical and chemical industries. Producing large quantities of light olefin material in an economical manner, therefore, is a focus in the petrochemical industry.

Acetylene can be used to make a variety of useful products such as ethylene and propylene. From recent methods of producing olefins, one method includes passing a hydrocarbon feedstock into a supersonic or pyrolytic reactor and accelerating it to supersonic speed to provide kinetic energy that can be transformed into heat to enable an endothermic pyrolysis reaction to occur. The hydrocarbon feedstock that can be used in the supersonic reactor includes methane. Pyrolysis of methane feeds to form acetylene and other useful products supersonic reactor, requires large amounts of heat to be produced in the supersonic reactor to provide the heat of reaction for the endothermic pyrolysis reactions. In order to generate a large amount of heat, a large amount of fuel is consumed. The reactor effluent from the supersonic reactor is separated in the downstream separation zone or product recovery section of the supersonic reactor including various columns and associated equipment in between.

Further, there are some processes including, but not limited to, steam cracking processes producing fuel gas stream having surplus methane. Usually, in a refinery all the fuel gases streams that are produced end up in a common refinery header. Typically, these fuel gas streams comprise methane which also ends up in the common refinery header. Generally, these gases are not further utilized in the process and withdrawn. Furthermore, these processes also include product separation section wherein the product streams are separated from the byproducts including fuel gas streams.

Accordingly, it is desirable to provide new apparatuses and processes for providing cost benefits in terms of lower capital and operational expenditures. Also, there is a need for an alternative approach to maximize recovery of hydrocarbons from such processes. Other desirable features and characteristics of the present subject matter will become apparent from the subsequent detailed description of the subject matter and the appended claims, taken in conjunction with the accompanying drawings and this background of the subject matter.

### BRIEF SUMMARY

Various embodiments contemplated herein relate to integrated processes and apparatuses for producing acetylene. The exemplary embodiments taught herein provide an integrated process for producing acetylene by integrating various processes.

In accordance with an exemplary embodiment, an integrated process is provided for producing acetylene. The integrated process comprises recovering a fuel gas stream from a product recovery unit. A gas stream comprising methane may be separated from the fuel gas stream in the product recovery unit. A fuel and an oxidizer are combusted in a combustion zone of a pyrolytic reactor to create a combustion gas stream, wherein the pyrolytic reactor is integrated with the product recovery unit via the gas stream comprising methane. Velocity of the combustion gas stream is accelerated from subsonic to supersonic in an expansion zone of the pyrolytic reactor to provide a supersonic combustion gas stream. A light hydrocarbon stream comprising all or a first portion of the gas stream comprising methane may be injected into the supersonic combustion gas stream to create a mixed stream including the light hydrocarbon stream. The velocity of the mixed stream is transitioned from supersonic to subsonic in a reaction zone of the pyrolytic reactor to produce a reaction mixture comprising acetylene, methane, carbon oxides, and hydrogen. The reaction mixture is separated to provide an acetylene stream.

In accordance with another exemplary embodiment, an integrated process is provided for producing acetylene. The integrated process comprises combusting a fuel and an oxidizer in a combustion zone of a pyrolytic reactor to create a combustion gas stream. The velocity of the combustion gas stream may be accelerated from subsonic to supersonic in an expansion zone of the pyrolytic reactor. A light hydrocarbon stream may be injected into the supersonic combustion gas stream to create a mixed stream comprising the light hydrocarbon. The velocity of the mixed stream is transitioned from supersonic to subsonic in a reaction zone of the pyrolytic reactor to produce a reaction mixture comprising acetylene, methane, carbon oxides, and hydrogen. The reaction mixture is passed to a product recovery unit integrated with the pyrolytic reactor. In the integrated product recovery unit, the reaction mixture may be separated to provide an acetylene stream and a fuel gas stream comprising methane, carbon oxides and the hydrogen.

In accordance with yet another exemplary embodiment, an integrated process is provided for producing acetylene. The integrated process comprises combusting a fuel and an oxidizer in a combustion zone of a pyrolytic reactor to create a combustion gas stream. Velocity of the combustion gas stream may be accelerated from subsonic to supersonic in an expansion zone of the pyrolytic reactor. A light hydrocarbon stream is injected into the supersonic combustion gas stream to create a mixed stream including the light hydrocarbon. Velocity of the mixed stream is transitioned from supersonic to subsonic in a reaction zone of the pyrolytic reactor to produce a reaction mixture comprising acetylene, methane, carbon oxides, and hydrogen. In a separation zone of the pyrolytic reactor, the reaction mixture may be separated into an acetylene stream and a byproduct stream comprising the methane, carbon oxides and the hydrogen. The byproduct stream is passed to a product recovery unit integrated with the pyrolytic reactor, wherein the pyrolytic reactor is integrated with the product recovery unit via the byproduct stream. The byproduct stream is separated in the product recovery unit to provide a gas stream comprising methane. All or a first portion of the gas stream comprising methane may be injected into the supersonic combustion gas stream.

The integrated process of the present disclosure envisages integration of a methane pyrolysis process with any process producing surplus methane which is usually withdrawn as fuel stream from the process. Usually, in a refinery all the fuel gases stream that are produced end up in a common refinery header. Typically, these fuel gas streams also comprise methane which also ends up in the common refinery header. The present integrated process envisages utilizing these fuel gas streams with surplus methane and integrating such processes with the methane pyrolysis process to enhance overall recovery of the process. Also, the current integrated process envisages reduction of overall CAPEX of the process by integrating and using purification equipment between the processes.

These and other features, aspects, and advantages of the present disclosure will become better understood upon consideration of the following detailed description, drawings and appended claims.

### BRIEF DESCRIPTION OF THE DRAWING

The various embodiments will hereinafter be described in conjunction with the following FIGURES, wherein like numerals denote like elements.

FIG. 1 is a schematic diagram of an integrated process and an apparatus for producing acetylene in accordance with an exemplary embodiment.

FIG. 2 is an illustration of an exemplary embodiment of a pyrolytic reactor in accordance with the integrated process and the apparatus of the present disclosure.

FIG. 3 is a schematic diagram of an integrated process and an apparatus for producing acetylene in accordance with yet another exemplary embodiment.

FIG. 4 is a schematic diagram of an integrated process and an apparatus for producing acetylene in accordance with still another exemplary embodiment.

### DEFINITIONS

As used herein, the term “column” or “tower” means a distillation column or columns for separating one or more components of different volatilities. Unless otherwise indicated, each column includes a condenser on an overhead of the column to condense the overhead vapor and reflux a portion of an overhead stream back to the top of the column. Also included is a reboiler at a bottom of the column to vaporize and send a portion of a bottom stream back to the bottom of the column to supply fractionation energy. Feeds to the columns may be preheated. The top pressure is the pressure of the overhead vapor at the outlet of the column. The bottom temperature is the liquid bottom outlet temperature. Overhead lines and bottom lines refer to the net lines from the column downstream of the reflux or reboil to the column. Alternatively, a stripping stream may be used for heat input at the bottom of the column.

As used herein, the term “stream” can include various hydrocarbon molecules and other substances.

As used herein, the term “overhead stream” can mean a stream withdrawn in a line extending from or near a top of a vessel, such as a column.

As used herein, the term “bottoms stream” can mean a stream withdrawn in a line extending from or near a bottom of a vessel, such as a column.

The term “Cₓ₋” wherein “x” is an integer means a hydrocarbon stream with hydrocarbons have x and/or less carbon atoms and preferably x and less carbon atoms.

The term “Cₓ₊” wherein “x” is an integer means a hydrocarbon stream with hydrocarbons have x and/or more carbon atoms and preferably x and more carbon atoms.

As used herein, the term “passing” includes “feeding” and “charging” and means that the material passes from a conduit or vessel to an object.

As used herein, the term “portion” means an amount or part taken or separated from a main stream without any change in the composition as compared to the main stream. Further, it also includes splitting the taken or separated portion into multiple portions where each portion retains the same composition as compared to the main stream.

As used herein, the term “unit” or “zone” can refer to an area including one or more equipment items and/or one or more sub-units. Equipment items can include one or more reactors or reactor vessels, heaters, separators, drums, exchangers, pipes, pumps, compressors, and controllers. Additionally, an equipment item, such as a reactor, dryer, or vessel, can further include one or more units or sub-units.

The term “communication” means that material flow is operatively permitted between enumerated components.

The term “steam cracker” or “steam cracking unit” as used herein is also known more generally as a thermal pyrolysis unit. Steam, although optional, is typically added inter alia to reduce hydrocarbon partial pressure, to control residence time, and to minimize coke formation. The steam to the steam cracking unit may be superheated, such as in the convection section of the pyrolysis unit, and/or the steam may be sour or treated process steam.

As used herein, the term “boiling point” means the boiling points of material that are more conveniently determined by gas chromatography simulated distillation methods, ASTM D-2887 and ASTM D-7169.

The following detailed description is merely exemplary in nature and is not intended to limit the various embodiments or the application and uses thereof. Furthermore, there is no intention to be bound by any theory presented in the preceding background or the following detailed description. The Figures have been simplified by the deletion of a large number of apparatuses customarily employed in a process of this nature, such as vessel internals, temperature and pressure controls systems, flow control valves, recycle pumps, etc. which are not specifically required to illustrate the performance of the invention. Furthermore, the illustration of the process of this invention in the embodiment of a specific drawing is not intended to limit the invention to specific embodiments set out herein.

### DETAILED DESCRIPTION

As depicted, process flow lines in the figures can be referred to, interchangeably, as, e.g., lines, pipes, branches, distributors, streams, effluents, feeds, products, portions, catalysts, withdrawals, recycles, suctions, discharges, and caustics.

The methane pyrolysis process uses a pyrolytic reactor or supersonic reactor to produce acetylene. The feedstock for the pyrolytic reactor includes methane. The pyrolytic reactor converts methane to acetylene at very high temperatures. The reactor effluent contains mainly acetylene, methane, carbon oxides, hydrogen, water, and some heavier compounds. The water is removed in a quench tower before the cracked gases are sent to a compressor. The compressed gas is sent to an absorption unit to absorb the acetylene in a solvent. The compressed gas which is not absorbed contains methane, carbon oxides, and hydrogen. Further, some processes produce by-product gases comprising methane. Usually the by-product gases form part of the fuel gas stream from the processes and are withdrawn without further utilization in the process.

Usually, in a refinery all of the fuel gases stream that are produced end up in a common refinery header. Typically, these fuel gas streams may comprise methane which also ends up in the common refinery header.

The present integrated process provides integration of the methane pyrolysis process with such chemical processes or the refinery operations producing by-product gases comprising methane. The present integrated process provides various benefits including (i) upgrading excess low-value methane by-product streams to higher value products by the methane pyrolysis process, (ii) use of the by-product syngas from a gas to chemicals process as fuel for the steam cracking unit and other ancillary process units, (iii) integration of the product recovery and purification equipment between the processes to reduce CAPEX (iv) generation of high-value acetylene and acetylene derivatives including ethylene by using low-value methane by-product stream to increase operating margin and profit, and (v) reduction of syngas treating OPEX and CAPEX for pyrolytic reactor or gas to chemicals reactor by integration of the product recovery and purification equipment between the processes, and other ancillary process units.

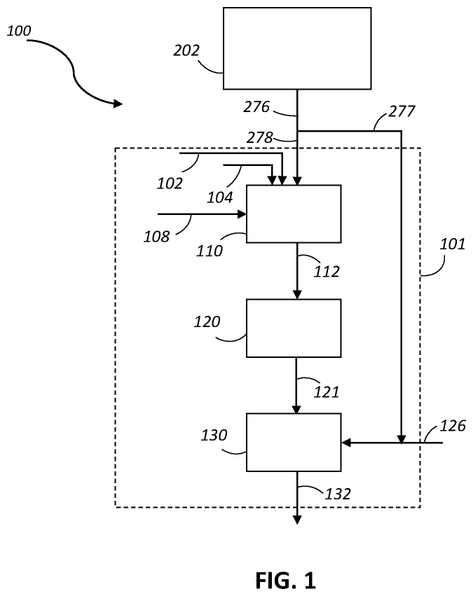

An integrated process for producing acetylene is addressed with reference to a process and an apparatus 100 according to an exemplary embodiment as shown in FIG. 1. A pyrolytic reactor is illustrated as 101 in FIG. 1. As shown, a product recovery unit 202 of an exemplary process is integrated with the pyrolytic reactor 101. As described herein after in detail, the product recovery unit 202 of the process is in fluid communication with the pyrolytic reactor 101 via line 276. A gas stream comprising methane may be recovered as by-product in the product recovery unit 202 and passed to the pyrolytic reactor 101 in line 276. In an embodiment, the pyrolytic reactor 101 is integrated with the product recovery unit 202 via the gas stream comprising methane in line 276. As shown, the gas stream comprising methane in line 276 may be passed to the pyrolytic reactor 101 via line 277 and/or via line 278. In an exemplary embodiment, a first portion of the gas stream comprising methane may be passed to the pyrolytic reactor 101 in line 277. In another exemplary embodiment, a second portion of the gas stream comprising methane may be passed to the pyrolytic reactor 101 in line 278.

In the integrated process as shown in FIG. 1, a light hydrocarbon stream in line 126 may be converted into acetylene in the pyrolytic reactor 101. In one non-limiting example, the light hydrocarbon stream may comprise methane. In an exemplary embodiment, the light hydrocarbon stream in line 126 may comprise all or the first portion of the gas stream comprising methane in line 277. The light hydrocarbon stream in line 126 may be all the first portion of the gas stream comprising methane in line 277. In an exemplary embodiment, the first portion of the gas stream comprising methane in line 277 may comprise from about 0 to about 100 vol % of the gas stream comprising methane in line 276. The light hydrocarbon stream in line 126 may additionally include the first portion of the gas stream comprising methane in line 277. In an embodiment as shown in FIG. 1, the pyrolytic reactor 101 may comprise a combustion zone (not shown), an expansion zone 120, and a reaction zone 130. The pyrolytic reactor 101 may be alternatively called as a gas to chemicals (GTC) reactor. The pyrolytic reactor 101 may receive a fuel in a fuel line 102. The pyrolytic reactor 101 may receive an oxidizer (oxygen) via an oxygen rich stream in line 104. The pyrolytic reactor 101 may also receive a diluent in line 108. In an embodiment, a portion of the gas stream comprising methane in line 276 may also be passed to the pyrolytic reactor 101 as fuel. Although not shown, a portion of the gas stream comprising methane in line 276 may be combined with the fuel in line 102 to provide a combined fuel stream. The combined fuel stream may be passed to the pyrolytic reactor 101 as fuel. In an exemplary embodiment, the second portion of the gas stream comprising methane in line 278 may be passed to the pyrolytic reactor 101 as fuel. The fuel in line 102 and/or in line 278, the diluent stream in line 108, and the oxidizer in line 104 may be combusted in the combustion zone to provide a combustion gas stream in line 112. The combustion gas stream in line 112 may enter the expansion zone 120 and flows to the reaction zone 130 in line 121. The velocity of the combustion gas stream in line 112 transitions from subsonic (i.e., less than Mach 1) to supersonic (i.e., greater than Mach 1) within the expansion zone 120. A reactor effluent stream comprising acetylene may exit the reaction zone 130 in line 132.

A pyrolytic reactor outlet stream in line 132 produced by the pyrolytic reactor 101 may include acetylene, ethylene, hydrogen, methane, carbon monoxide, carbon dioxide, and carbon particulates.

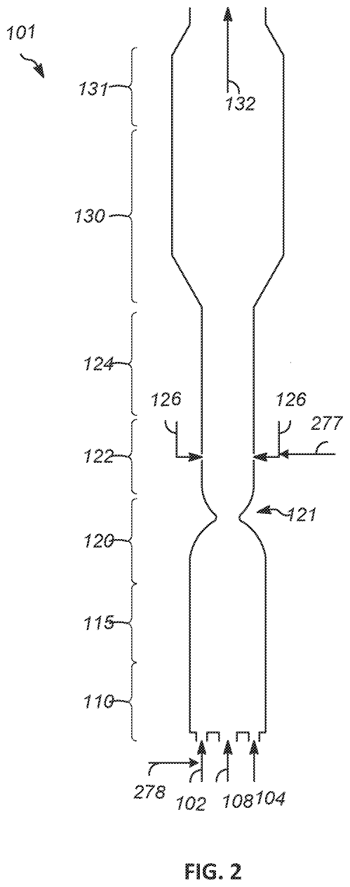

In an exemplary embodiment, the pyrolytic reactor 101 is illustrated in FIG. 2. A longitudinal cross section of an exemplary pyrolytic reactor 101 is shown in FIG. 2. In one exemplary embodiment, the pyrolytic reactor 101 may be tubular (i.e., the transverse cross section is circular). The high temperatures necessary for the formation of acetylene as well as controlled residence time and rapid quenching can be achieved in the pyrolytic reactor 101. The diluent stream in line 108, the fuel in fuel line 102, and the oxidizer (e.g., oxygen) in line 104 may be injected in a fuel injection zone 110 at the proximal end of the pyrolytic reactor 101. The second portion of the gas stream comprising methane in line 278 may be passed along with the fuel in fuel line 102 into the fuel injection zone 110. Alternatively, the second portion of the gas stream comprising methane in line 278 may be passed separately to the fuel injection zone 110. In an exemplary embodiment, the second portion of the gas stream comprising methane in line 278 may comprise from about 0 to about 100 vol % of the gas stream comprising methane in line 276.

The light hydrocarbon stream in line 126 may be heated in the pyrolytic reactor 101 to a temperature at which the formation of acetylene is thermodynamically favored over that of methane. Additional energy must be provided to a reaction mixture to satisfy the endothermic reaction for the formation of acetylene. After a residence time sufficient to result in the desired acetylene formation, the reaction mixture may be quickly quenched to freeze the reaction in order to prevent the acetylene from cracking into hydrogen and carbon and reforming as methane. A fuel and oxidizer may be combusted to create a high temperature (e.g., >1227° C. (1500 K)) and high speed (e.g., >Mach 1) combustion gas, in order to favor acetylene formation. Next, a sufficient amount of reaction enthalpy is provided to satisfy the requirement of 377 kJ/mol for the formation of acetylene. If additional energy is not provided, the endothermic nature of the acetylene formation may drive the temperature below 1227° C. (1500 K). Finally, the reaction mixture is quickly cooled at a rate faster than the rate at which the acetylene can decompose into hydrogen and carbon and subsequently reform as methane. This quick cooling process is sometimes referred to as “freezing” the reaction when the amount of acetylene is high. It is desirable to initiate the freezing step at the stage of maximum acetylene formation (i.e., the point of thermodynamic equilibrium) and to complete the freezing step as quickly as possible to prevent the decomposition of any acetylene.

In one embodiment, the fuel with the second portion of the gas stream comprising methane in line 102, the diluent stream in line 108, and the oxidizer in line 104 may be heated to a temperature of about 400° C. (674 K) to about 800° C. (1074 K), or to a temperature of about 200° C. (474 K) to about 1000° C. (1274 K). The fuel in line 102 may be selected from hydrogen or methane. In an exemplary embodiment, the fuel is hydrogen. The oxidizer may be oxygen. The ratio of hydrogen to oxygen may be a 3/1 molar ratio. However, other suitable molar ratios of hydrogen to oxygen may also be used.

In some embodiments, the fuel with the second portion of the gas stream comprising methane in line 102, the diluent stream in line 108, and the oxidizer in line 104 may be mixed prior to injection into the fuel injection zone 110. In some embodiments, the fuel with the second portion of the gas stream comprising methane in line 102, diluent stream in line 108, and the oxidizer in line 104 may be injected into the fuel injection zone 110 and get mixed by the turbulent conditions within the fuel injection zone 110. In an exemplary embodiment, steam may be injected as the diluent stream in line 108 into the fuel injection zone 110. However, any suitable diluent may be injected as the diluent stream in line 108 into the fuel injection zone 110.

The fuel along with the second portion of the gas stream comprising methane in line 102, the diluent stream in line 108, and the oxidizer in line 104 may be combusted in the combustion zone 115 to create a combustion gas stream. The resulting combustion gas stream may be heated to a high temperature by the combustion reaction. In some embodiments, the temperature of the combustion gas stream may be from about 2227° C. (2500 K) to about 3227° C. (3500 K) in the combustion zone 115. In other embodiments, the temperature of the combustion gas stream may reach about 1727° C. (2000 K) to about 3727° C. (4000 K) in the combustion zone 115.

The combustion zone 115 may be operated at a pressure of about 200 kPa to about 1000 kPa (2 to 10 bar) or about 120 kPa to about 2000 kPa (1.2 bar to 20 bar). The pressure within the combustion zone 115 propels the combustion gas stream toward the distal end of the pyrolytic reactor 101 at a high velocity. In an embodiment, the velocity of the combustion gas stream at the distal end of the combustion zone 115 may be below supersonic speed (i.e., less than Mach 1). Since the combustion zone 115 operates at a relatively higher pressure, the second portion of the gas stream comprising methane in line 278 may be compressed before passing to the fuel injection zone 110. The second portion of the gas stream comprising methane in line 278 may be compressed by passing the second portion in line 278 to a pump to provide a compressed gas stream comprising methane. The compressed gas stream comprising methane may be passed to combustion zone 115 via the fuel injection zone 110 along with the fuel in fuel line 102. If required, the fuel in fuel line 102 may also be compressed.

The subsonic combustion gas stream may enter an expansion zone 120 and flows through a convergent-divergent nozzle 121. The velocity of the combustion gas stream may be accelerated from subsonic to supersonic in the expansion zone 120 to provide a supersonic combustion gas stream. The convergent-divergent nozzle 121 transforms a portion of the thermal energy in the combustion gas stream into kinetic energy, resulting in a sharp increase in the velocity of the combustion gas stream. The velocity of the combustion gas stream transitions from subsonic (i.e., less than Mach 1) to supersonic (i.e., greater than Mach 1) within the expansion zone 120. In one embodiment, at the distal end of the expansion zone 120, the temperature of the combustion gas stream can be about 1227° C. (1500 K) to about 3274° C. (3000 K). In another embodiment, at the distal end of the expansion zone 120, the average velocity of the combustion gas stream (across a transverse cross section) can be greater than Mach 1. In yet another embodiment, the combustion gas stream may have an average velocity of about Mach 2 or above.

As shown in FIG. 2, the light hydrocarbon stream in line 126 may be injected into the supersonic combustion gas stream in a feedstock injection zone 122 to create a mixed stream. The feedstock may be injected at a temperature of about 427° C. (700 K) to 927° C. (1200 K) or about 27° C. (300 K) to 1727° C. (2000 K). In an exemplary embodiment, the light hydrocarbon stream comprises methane. In an embodiment, concentration of methane in the light hydrocarbon stream in line 126 may range from about 65 mol % to about 100 mol %, or from about 80 mol % to about 100 mol %, or about 90 mol % to about 100 mol %. In another exemplary embodiment, the light hydrocarbon stream in line 126 may comprise all or a first portion of the gas stream comprising methane in line 277. The light hydrocarbon stream in line 126 may be all the first portion of the gas stream comprising methane in line 277. Accordingly, the light hydrocarbon stream comprising all or the first portion of the gas stream comprising methane may be injected in line 126 into the supersonic combustion gas stream to create a mixed stream including the light hydrocarbon stream. In another embodiment, the light hydrocarbon stream in line 126 may additionally include the first portion of the gas stream comprising methane in line 277. Since, the first portion of the gas stream comprising methane in line 277 is passed via the feedstock injection zone 122 to the reaction zone 130 which typically operates at a lower pressure, the first portion of the gas stream comprising methane in line 277 can be passed to the reaction zone 130 without compressing as compared to the second portion of the gas stream comprising methane in line 278. However, if necessary, a compressor can also be used to boost the pressure the light hydrocarbon stream in line 126 and/or the first portion of the gas stream comprising methane in line 277 which is passed as a feed to the pyrolytic reactor 101.

A combined stream comprising the combustion gas stream, the light hydrocarbon stream in line 126, along with the first portion of the gas stream comprising methane in line 277 may enter a mixing zone 124 where the combined stream may be mixed as a result of the turbulent flow in the stream to provide a mixed stream. The mixed stream may comprise the combustion gas stream, the light hydrocarbon stream in line 126, along with the first portion of the gas stream comprising methane in line 277. In an embodiment, oblique or normal shockwaves can be used to assist the mixing in the mixing zone 124.

The mixed stream may enter the reaction zone 130. In an embodiment, the velocity of the mixed stream may remain at supersonic velocities within the reaction zone 130. In an exemplary embodiment, the first portion of the gas stream comprising methane in line 277 may be injected into the reaction zone 130 via the mixing zone 124. In an embodiment, the first portion in line 277 may range from about 0 to about 100 vol % of the gas stream comprising methane in line 276.

Shocks may be created in the reaction zone 130 by adjusting the backpressure of the reactor 101. Shocks will reduce the velocity and convert a portion of kinetic energy into thermal energy. The combined stream or the mixed stream may be then reduced to subsonic flow and quenched in a quenching zone 131.

In an embodiment, the velocity of the mixed stream transitions from supersonic to subsonic within the reaction zone 130 to produce a reaction mixture comprising acetylene, methane, carbon oxides, and hydrogen. At this transition point, a shockwave may be formed, which results in a nearly instantaneous increase in the pressure and temperature of the mixed stream. In an embodiment, the temperature of the mixed stream immediately upstream of the shock wave may be about 1227° C. (1500 K) to about 2027° C. (2300 K), as compared to about 1327° C. (1600 K) to about 2527° C. (2800 K) immediately downstream of the shockwave. The conditions in the mixed stream downstream of the shockwave are favorable to the formation of acetylene. Thus, the pyrolytic reactor 101 may be called a shock wave reactor (SWR).

In some embodiments, a shock train may be formed at the point where the stream transitions from supersonic to subsonic flow. A shock train is a series of weak shock waves that propagate downstream from the supersonic to subsonic transition point. Whereas, a single shockwave will heat the mixture nearly instantaneously (at the location of the shockwave), a shock train may heat the mixture more gradually. Each shock wave in the shock train may increase the temperature of the stream. The mixed stream may be increased to a temperature sufficient to favor the formation of acetylene and to provide enough energy to satisfy the endothermic reaction.

A reactor effluent stream comprising the reaction mixture may exit the reaction zone 130 and enter the quenching zone 131 to rapidly cool the reactor mixture. The quenching zone 131 may comprise at least one injection nozzle to spray the reactor effluent stream with water. A cooled reactor effluent stream may be withdrawn in line 132. The cooled reactor effluent stream comprising the reaction mixture in line 132 may be separated to provide an acetylene stream.

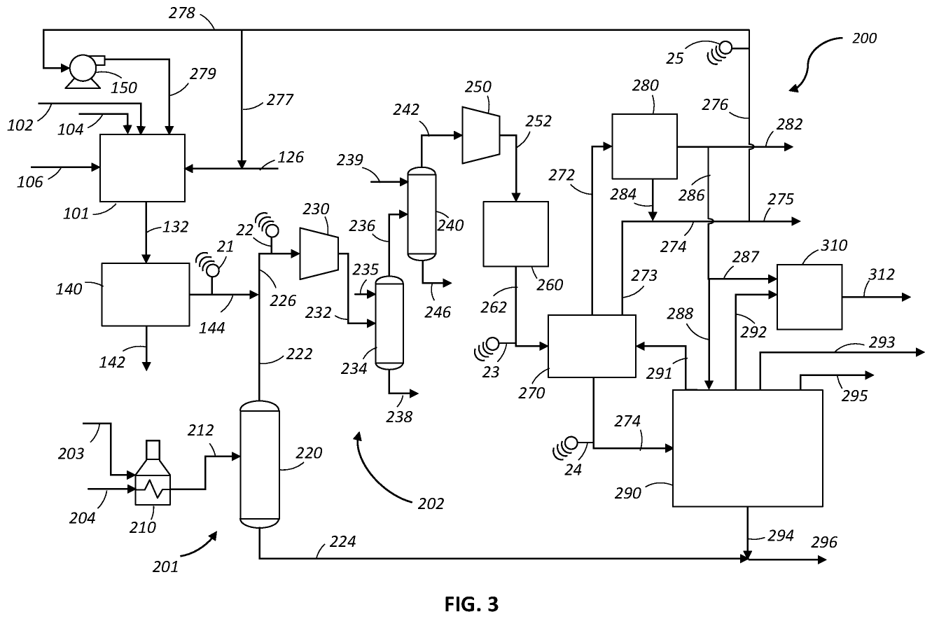

In an embodiment, the pyrolytic reactor 101 is integrated with a steam cracking process having a steam cracking unit 201 as shown in FIG. 3. The steam cracking unit 201 may comprise a product recovery unit 202. A fuel gas stream may be recovered in the product recovery unit 202 as a by-product from the product recovery unit 202. A gas stream comprising methane may be separated from the fuel gas stream and passed to the pyrolytic reactor 101 in line 276 as described herein above. Quenching the reactor effluent prevents further reaction in the reactor effluent stream and also removes particulate matter present in the reactor effluent stream. After quenching, the reactor effluent stream in line 132 may be passed to a separation zone 140 for separating an acetylene stream. The separation zone 140 may be included within the pyrolytic reactor 101 or used as a separate unit. In an exemplary embodiment, the pyrolytic reactor 101 includes the separation zone 140. The separation zone 140 may comprise an absorber wherein the reactor effluent stream in line 132 may be compressed and contacted with a solvent. The solvent absorbs acetylene, and the stream comprising solvent and acetylene is separated and recovered to provide an acetylene stream in line 142. The acetylene stream in line 142 can be directly used for downstream conversion processes. A byproduct stream that does not absorb in the solvent is also separated in the separation zone 140. The byproduct stream from the separation zone 140 comprises methane, carbon oxides, and hydrogen. The byproduct stream may exit the separation zone 140 in line 144. Alternatively, the byproduct stream in line 144 may be called a syn gas stream. Suitable solvents that may be used for absorbing acetylene may include n-methyl-2-pyrrolidone, dimethylformamide, acetone, tetrahydrofuran, dimethylsulfoxide, monomethylamine, and combinations thereof.

The byproduct stream in line 144 from the separation zone 140 may comprise recoverable methane. Accordingly, applicants' process provides for separation of this recoverable methane from the byproduct stream in line 144 by integrating the pyrolytic reactor 101 via the byproduct stream in line 144 with another process. By such integration, a product recovery and purification unit of a downstream process may be utilized thereby reducing syngas treating OPEX and CAPEX for pyrolytic reactor 101. The methane so recovered can be utilized further as per the requirement. In an exemplary embodiment, the recovered methane may be recycled to the pyrolytic reactor 101 as the fuel and/or the feedstock to the reaction zone 130.

In an exemplary embodiment, the pyrolytic reactor 101 may be integrated with the steam cracking process 201 via the byproduct stream in line 144. In the steam cracking process, steam is usually mixed with a feed comprising high molecular weight hydrocarbons. A mixed stream is passed to a cracking reactor to reduce the hydrocarbon partial pressure and thereby enhance olefin yield. The presence of steam also reduces the formation and deposition of carbonaceous material in the cracking reactors. The process may also be referred to as a pyrolysis process. The feed that is fed to a steam cracking unit can be quite diverse and can be chosen from a variety of petroleum fractions. Accordingly, the configuration of steam cracking unit may also vary depending upon the feed and separation thereafter.

In an embodiment, the feed to a steam cracking unit may be characterized by a boiling point range falling within the naphtha boiling point range of about 36° C. to about 195° C. Naphtha is a gasoline range boiling hydrocarbon having a carbon range of C₅ to C₁₂. The feed to the steam cracking unit may comprise C₂ to C₄₀ hydrocarbons, or C₂ to C₃₀ hydrocarbons, or C₂ to C₂₀ hydrocarbons. In another embodiment, the feed to the steam cracking unit may comprise C₂ to C₄ hydrocarbons.

Within the steam cracking unit, the feed stream contacts steam under conditions, e.g., temperature and pressure, effective to convert at least a portion of the feed stream to olefins e.g. ethylene and propylene, which exits the steam cracking unit in a steam cracking effluent stream. The steam cracking effluent stream may contain a variety of contaminants and C₄₊ components in addition to ethylene and propylene. Therefore, separation of these various hydrocarbon components is necessary in order to yield product of desired grade. For example, upon exiting the steam cracking unit, the steam cracking effluent stream preferably may be cooled in an indirect quench unit to form a cooled steam cracking effluent stream. The cooled steam cracking effluent stream may be directed to one or more fractionation units for separation.

The one or more fractionation units usually separates the cooled steam cracking effluent stream into one or more of a light hydrocarbon stream containing mostly C₅₋ components, a gasoline stream (also referred to as pyrolysis gasoline or pygas) containing mostly C₆ components and optionally water, and a fuel oil stream containing C₉₊ hydrocarbon components. Specifically, the fuel oil stream contains at least a majority of the fuel oil that was present in the cooled steam cracking effluent stream. The gasoline stream contains at least a majority of the gasoline that was present in the cooled steam cracking effluent stream.

The steam cracking unit may also include a quench tower. In the quench tower, the light hydrocarbon stream, or a portion thereof, contacts a quench medium, preferably water, under conditions effective to separate readily condensable components from non-readily condensable components. Preferably, two phases form in the bottom of the quench tower, a pygas stream which contains mostly C₆ hydrocarbons, and a heavier water containing stream. A portion of the water containing stream optionally is cooled in one or more heat exchangers and reintroduced into the quench tower via one or more quench medium inlets. Nonreadily condensable components such as ethylene and propylene are withdrawn from the quench tower via a quench overhead Stream. The majority of the C₅₋ components preferably are yielded from the quench tower in the quench overhead Stream.

The quench overhead stream from the quench tower may be directed to a compression stage comprising one or more compressors. The compression stage may comprise one or more heat exchangers for cooling intermediate condensed streams, and one or more knockout drums to separate condensed components from noncondensed components. A compressed effluent stream from the compression system may be directed to a caustic wash unit for removal of carbon dioxide therefrom.

In the caustic wash unit, the compressed stream may be contacted with caustic, e.g., sodium hydroxide, under conditions effective to remove carbon dioxide therefrom. Sodium bicarbonate (NaHCO3) optionally is formed as a byproduct of the carbon dioxide removal process. Thus, the caustic wash unit forms an overhead CO₂ depleted stream and a spent caustic bottoms Stream. Optionally, the CO₂ depleted stream or a portion thereof is compressed in one or more additional stages. Also, the CO₂ depleted stream or a portion thereof optionally may be directed to a drying unit, preferably a molecular sieve drying unit, wherein the CO₂ depleted stream contacts a water removal medium under conditions effective to remove water from the CO₂ depleted stream. Preferably, the water removal medium comprises a molecular sieve particle adapted to selectively adsorb water molecules. That is, the drying unit removes water from the carbon dioxide depleted stream to form a dry product stream comprising ethylene, propylene and optionally light ends such as hydrogen, carbon monoxide and methane and/or C₄₊ hydrocarbons. The dry product stream, or a portion thereof, may be directed to a separation unit that is adapted to separate the dry stream into two or more of its individual components.

In an exemplary embodiment, an integrated process for producing acetylene is addressed with reference to a process and an apparatus 200 according to an embodiment as shown in FIG. 3. As shown, the pyrolytic reactor 101 is integrated with a steam cracking process via the byproduct stream in line 144. The steam cracking process as shown in FIG. 3 may comprise the steam cracking unit 201 and the product recovery unit 202. A hydrocarbonaceous feedstock in line 204 may be passed to the steam cracking unit 201. The hydrocarbonaceous feedstock in line 204 may be passed to the preheat zone 210 of the steam cracking unit 201. In an exemplary embodiment, the hydrocarbonaceous feedstock to the steam cracking unit 201 may be selected from one or more of naphtha, kerosene, condensate, atmospheric gas oil, vacuum gas oil, hydrocrackate, and crude oil. The preheat zone 210 may comprise a preheat region and a cracking zone. In the preheat region, the feedstock 204 may be heated to form a heated feedstock stream having a temperature from about 93° C. (366 K) to about 232° C. (505 K). Steam in line 203 may also be passed to the preheat zone 210. The steam in line 203 may be mixed with the heated feedstock stream and thereafter directed to the cracking zone. In the cracking zone, the steam containing heated feedstock may be further heated under conditions effective to “crack” or “pyrolyze” the hydrocarbonaceous feedstock in the presence of steam and produce a steam cracked effluent stream in line 212 comprising fuel oil, gas oil, pyrolysis gasoline, and C₅₋ hydrocarbon components (including methane, ethylene and propylene). The steam cracked effluent stream in line 212 may be quenched before passing to the product recovery unit 202 of the steam cracking process.

The feed to the steam cracking unit 201 is usually rich in isobutane (LPG) and light naphtha which tends to produce a surplus of by-product methane than the fuel needs of the steam cracking unit. This surplus by-product methane is present in the steam cracked effluent stream in line 212. The steam cracked effluent stream in line 212 tends to be relatively rich in methane, may contain some hydrogen, but has low concentration of carbon dioxide and carbon monoxide. Applicants integrated process may also utilize this by-product methane to provide more valuable acetylene and/or acetylene derivatives including ethylene. Due to the high price of acetylene and/or acetylene derivatives relative to methane, applicants' integrated process can increase the overall operating profit of the steam cracking operator.

The steam cracked effluent stream in line 212 may be quenched to cool the effluent stream in line 212. Accordingly, effluent stream in line 212 may be quenched in a quench zone 220 to cool the effluent stream in line 212. In the quench zone 220, the steam cracked effluent stream in line 212 may be separated to provide a cracked gas effluent stream containing light olefins in an overhead line 222 and a bottoms liquid stream comprising fuel oil by-products including pyrolysis gasoline in line 224. The cracked gas effluent stream in overhead line 222 may comprise C₂-C₄ olefins, methane, carbon oxide, and hydrogen. In an exemplary embodiment, the steam cracked effluent stream in line 212 may be passed to a two-stage quench zone 220 to cool the effluent stream and to separate the light olefin products from any heavy hydrocarbon or pygas (pyrolysis gasoline). Although not shown, the two-stage quench zone 220 may comprise an oil quench tower and a water quench tower for quenching the steam cracked effluent stream in line 212. In the oil quench tower, an overhead stream comprising the light olefin and pyrolysis gasoline may be recovered in an overhead line. A bottoms stream comprising fuel oil by-products may be recovered in a bottoms line. The overhead stream in the overhead line may be passed to the water quench tower wherein the cracked gas effluent stream comprising light olefins may be recovered in an overhead line 222. A bottoms stream from the water quench tower comprising pyrolysis gasoline fraction or pygas may be recovered in line 224 from the two-stage quench zone 220. If needed, the bottoms stream in line 224 may be passed to a stabilizer column (not shown). In the stabilizer column, the pyrolysis gasoline may be separated from a gaseous stream. The gaseous stream may be withdrawn and recycled to the water quench tower of the two-stage quench zone 220. A bottoms stream comprising pyrolysis gasoline may be withdrawn from the stabilizer column in line 224. The bottoms stream comprising fuel oil by-products from the oil quench tower may be also be withdrawn in the bottoms stream in line 224. Accordingly, the bottoms stream comprising fuel oil by-products from the oil quench tower and the bottoms stream from the water quench tower may be combined and withdrawn in line 224.

The cracked gas effluent stream in the overhead line 222 may be further separated into the product recovery unit 202 of the steam cracking process to provide a fuel gas stream comprising methane. As shown, the cracked gas effluent stream in the overhead line 222 may be combined with the byproduct stream in line 144 from the separation zone 140 of the pyrolytic reactor 101 to obtain a combined overhead stream in line 226. The combined overhead stream in line 226 may be separated into the product recovery unit 202 to recover hydrocarbons present therein and provide the gas stream comprising methane. The combined overhead stream in line 226 may be passed to a first stage compressor 230. Alternately, the byproduct stream in line 144 and the cracked gas effluent stream in the overhead line 222 may be passed separately to the first stage compressor 230 and compressed therein. The first stage compressor 230 may comprise one or more compression stages to form a compressed effluent stream in line 232. In an exemplary embodiment the first stage compressor 230 comprises three compression stages. After each stage of compression, the compressed stream may be cooled causing the condensation of heavier components which can be collected in one or more knock out drums (not shown) between compression stages.

The compressed effluent stream may be further treated to remove acid gases. In an exemplary embodiment, the compressed effluent stream in line 232 may be passed through a trayed or packed absorption column 234 where it is scrubbed by means of an absorbent liquid such as an aqueous solution fed by line 235 to remove acid gases including hydrogen sulfide and carbon dioxide by absorbing them into the aqueous solution. If the pressure of the pyrolytic reactor effluent stream is boosted to be at least equal to the absorption column 234 inlet pressure, the byproduct stream in line 144 from the separation zone 140 of the pyrolytic reactor 101 may be passed to the packed absorption column 234 inlet pressure. Accordingly, a pump can used to compress the byproduct stream in line 144 and a compressed byproduct stream can be passed to the packed absorption column 234. Suitable aqueous solutions may include lean amines such as alkanolamines, diethanolamine, monoethanolamine, and methyldiethanolamine. Other amines can also be used in place of or in addition to these amines. The packed absorption column 234 may be alternatively called an amine treater 234. In the packed absorption column 234, the lean amine contacts the compressed effluent stream in line 232 and absorbs acid gas contaminants such as hydrogen sulfide and carbon dioxide. The resultant effluent stream can be taken out from an overhead outlet of the absorption column 234 in an overhead line 236, and a rich amine (spent absorbent liquid) can be taken out from the bottoms at a bottom outlet of the absorption column 234 in a bottoms line 238. The spent absorbent liquid from the bottoms in line 238 may be regenerated and recycled back (not shown) to the absorption column 234 in line 235.

The resultant effluent stream in the overhead line 236 may be further cleaned by directing the resultant effluent stream in the overhead line 236 to a caustic scrubber 240 for removal of remnant entrained acid gases such as CO₂. The caustic scrubber 240 may comprise one or more beds of a caustic compound. Caustic compounds that can be used in the caustic scrubber 240 may include alkaline compounds. Examples of such alkaline compounds may include sodium hydroxide and potassium hydroxide. The caustic compound may be passed in line 239 into the caustic scrubber 240. A spent caustic compound may be withdrawn in line 246 from the bottoms of the caustic scrubber 240. Although not shown, a portion of the spent caustic compound in line 246 may be recycled to the caustic scrubber 240 in line 239 along with fresh caustic compound. The caustic scrubber 240 removes CO₂ and other entrained acid gases that may be present in the resultant effluent stream in the overhead line 236 of the spent absorbent column 234 and forms CO₂ depleted “sweetened” stream in line 242. Optionally, a water stream (not shown) may also be introduced into the caustic scrubber 240 in order to ensure that CO2 depleted sweetened stream in line 242 does not contain entrained caustic medium or contains a minimal amount of caustic medium.

The sweetened stream in line 242 may be compressed in a second stage compressor 250. The second stage compressor 250 may comprise one or more compression stages to form a compressed sweetened stream in line 252. In an exemplary embodiment, the second stage compressor 250 comprises two compression stages. After each stage of compression, the compressed stream may be cooled causing the condensation of heavier components which can be collected in one or more knock out drums (not shown) between compression stages. The compressed sweetened stream in line 252 may be directed to a drying unit 260.

A solid or a liquid drying unit 260 can be used to remove water and/or additional oxygenated hydrocarbons from the compressed sweetened stream in line 252. In an exemplary embodiment, a solid drying unit 260 may be used. In the solid drying unit 260, the compressed sweetened stream in line 252 may contact a solid adsorbent to remove water and oxygenated hydrocarbons to very low levels. Adsorption is useful for removing water and oxygenated hydrocarbons to very low concentrations, and for removing oxygenated hydrocarbons that are not normally removed by using other treatment systems. The solid drying unit 260 may comprise multiple adsorbent beds. Multiple beds allow for continuous separation without the need for shutting down the process to regenerate the solid adsorbent.

The use of solid adsorbent in the adsorbent beds of the solid drying unit 260 depends on the types of contaminants being removed. Solid adsorbent may include alumina, silica, molecular sieves, and alumino-silicates. Beds containing mixtures of these adsorbents or multiple beds having different adsorbent solids can be used in the solid drying unit 260 to remove water as well as a variety of oxygenated hydrocarbons from the compressed sweetened stream in line 252.

In another exemplary embodiment, a liquid drying unit 260 may be used. In the liquid drying unit 260, a water absorbent may be used to remove water from the compressed sweetened stream in line 252. The water absorbent can be any liquid effective in removing water from an olefin-containing stream.

After treating the compressed sweetened stream in line 252, the drying unit 260 forms a “dry stream”. In accordance with the present integrated process, the “dry stream” can be defined as a stream having a reduced amount of water or moisture as compared to the compressed sweetened stream in line 252. The dry stream in line 262 may be directed to downstream recovery unit for removal of the remaining components contained therein, as described in more detail below.

The dry stream in line 262 may be passed to a cold box 270 to separate the fuel gas stream and valuable hydrocarbons from the dry stream in line 262. The cold box 270 may operate at cryogenic conditions and employ a Joule Thompson effect and refrigeration to separate hydrogen from the dry stream in line 262. The cold box 270 may contain heat exchange steps associated with the separation of the dry stream in line 262 into a liquid stream in line 274 comprising C₂₊ hydrocarbons and the fuel gas stream. The fuel gas stream so produced can be further separated into a relatively hydrogen rich stream and a relatively methane rich methane stream. Hydrogen can be recovered from the hydrogen rich stream in line 272. Accordingly, a light gas stream in line 272 comprising hydrogen is produced in the cold box 270 which may be further processed to recover hydrogen. Also produced in the cold box 270 as a result of associated multiple cooling and flashing steps is a light recycle stream 273 comprising hydrogen and methane. The liquid stream in line 274 comprising C₂₊ hydrocarbons and the light gas stream in line 272 comprising hydrogen and methane are withdrawn from the cold box 270.

The light gas stream in line 272 at an adsorption pressure ranging from 275 kPa (40 psia) to about 2.7 MPa (390 psia) may be withdrawn and passed to a pressure swing adsorption (PSA) unit 280. The pressure swing adsorption unit 280 may contain one or more adsorbent selected from the group consisting of alumina, silica gel, activated carbon, molecular sieves, and mixtures thereof. The adsorbent in the PSA unit 280 may be selected for the adsorption of methane over hydrogen to provide an adsorber effluent stream in line 282 and a desorption effluent stream in line 284. The adsorber effluent stream in line 282 comprises hydrogen and is essentially free of methane. The adsorber effluent stream in line 282 is rich in hydrogen. The adsorber effluent stream in line 282 may comprise from about 97 to about 99 mol % hydrogen, or from about 99 to about 99.9 mol % hydrogen, or about at least 99.9 mol % hydrogen. The desorption effluent stream in line 284 may be withdrawn from the PSA unit 280. The desorption effluent stream in line 284 comprises methane. The desorption effluent stream in line 284 may comprise from about 30 to about 60 mol % methane or more. In an embodiment, the desorption effluent stream in line 284 may be combined with the light recycle stream 273 to provide a gas stream comprising methane in line 274. The gas stream comprising methane in line 274 may be termed as byproduct gas stream comprising methane recovered from the fuel recovery unit 202 of the steam cracking unit 201. Usually, the gas stream comprising methane obtained from the product recovery unit of the steam cracking process is withdrawn.

The present process provides integrating the product recovery unit 202 of the steam cracking unit 201 with the pyrolytic reactor 101 via the gas stream comprising methane in line 274. By such integration, the present integrated process utilizes the fuel gas stream which otherwise is withdrawn from the steam cracking process. Further, applicants' integrated process increases the recovery of methane in the gas stream comprising methane in line 274 by recovering the methane present in the byproduct stream 144 of the pyrolytic reactor in the product recovery unit 202 of the steam cracking unit 201. Thus, present integrated process provides a collective recovery of methane and more valuable hydrocarbons as described herein after in detail which is economical and greater than the methane recovery from individual processes.

In an exemplary embodiment, a portion of the fuel gas stream in line 276 may be passed to the pyrolytic reactor 101 for conversion to acetylene. A remaining portion of the fuel gas stream in line 275 may be withdrawn from the integrated process as flue gas stream. In another exemplary embodiment, the whole fuel gas stream in line 274 may be passed to the pyrolytic reactor 101 for conversion to acetylene.

The liquid stream in line 274 comprising C₂₊ hydrocarbons from the cold box 270 may be directed to downstream recovery unit for removal of the remaining and valuable components present therein. In an exemplary embodiment, liquid stream in line 274 may be passed to a separation zone 290 of the product recovery unit 202 of the steam cracking process for removal or separation of the remaining and valuable components from the liquid stream in line 274. As discussed earlier, the configuration of the separation zone 290 may vary depending upon the feed and separation of the desired components. Typically, the separation zone 290 comprises various columns for separation of C₂₊ hydrocarbons. In an exemplary embodiment, the separation zone 290 may comprise a demethanizer column, a deethanizer column, a depropanizer column, and a debutanizer column.

The liquid stream in line 274 may be first passed to a demethanizer column (not shown) to separate methane from the liquid stream in line 274. Methane may be recovered in an overhead stream. The overhead stream comprising the recovered methane in line 291 may be passed to the cold box 270. Alternatively, the overhead stream comprising methane in line 291 may be combined with the fuel gas stream in line 274. A bottoms stream comprising remnant hydrocarbons may be withdrawn from the demethanizer column.

The bottoms stream withdrawn from the demethanizer column may be passed to a deethanizer column (not shown). An overhead stream comprising C₂₋ hydrocarbons and a bottoms stream comprising C₃₊ hydrocarbons may be withdrawn from the deethanizer column. Some C₂₋ hydrocarbons may also remain present in the bottoms stream of the deethanizer column. The overhead stream comprising C₂₋ hydrocarbons may be withdrawn in line 292. As shown, the overhead stream comprising C₂₋ hydrocarbons in line 292 may be passed to an acetylene processing unit 310. The acetylene processing unit 310 may be used to recover acetylene from the integrated process or it can be used to convert acetylene to more valuable hydrocarbons including but not limited to ethylene.

In an exemplary embodiment, the acetylene processing unit 310 is a selective hydrogenation unit 310 for selective hydrogenation of C₂ hydrocarbons to more valuable ethylene. A portion of the hydrogen rich adsorber effluent stream in line 282 be withdrawn. The withdrawn hydrogen rich adsorber effluent stream portion may be passed to the selective hydrogenation unit 310 in line 287. In the selective hydrogenation unit 310, overhead stream comprising C₂₋ hydrocarbons in line 292 may be selectively hydrogenated in the presence of a catalyst to provide more valuable ethylene. An effluent stream comprising a relatively higher amount of ethylene compared to the overhead stream in line 292 may be withdrawn from the selective hydrogenation unit 310. The effluent stream may be passed to a fractionation column to recover valuable ethylene from the effluent stream in line 312. The fractionation column (not shown) may also be referred as a C₂ splitter. In the fractionation column, a side stream comprising ethylene may be withdrawn as a product stream from the fractionation column. A flue gas stream may be withdrawn from an overhead of the fractionation column. A bottoms stream comprising ethane may be withdrawn from the fractionation column which may be recycled or recovered for further use. Alternatively, the effluent stream comprising a relatively higher amount of ethylene compared to the overhead stream in line 292 may be withdrawn in line 312 for further processing.

The bottoms stream comprising C₃₊ hydrocarbons from the deethanizer column may be passed to a depropanizer column (not shown). In the depropanizer column, the bottoms stream comprising C₃₊ hydrocarbons may be separated into an overhead stream comprising C₃₋ hydrocarbons. A bottoms stream comprising C₄₊ may be withdrawn from the depropanizer column. Some amount of C₃₋ hydrocarbons may remain in the bottoms stream withdrawn from the depropanizer column. The overhead stream comprising C₃₋ hydrocarbons may be withdrawn and valuable hydrocarbons are recovered therefrom. In an exemplary embodiment, the overhead stream comprising C₃₋ hydrocarbons may be passed to another selective hydrogenation unit (not shown) of the separation zone 290 for selective hydrogenation of unstable compounds methyl acetylene and propadiene (MAPD). Another portion of the hydrogen rich adsorber effluent stream in line 282 may be withdrawn. The withdrawn hydrogen rich adsorber effluent stream portion may be passed to the selective hydrogenation unit of the separation zone 290 in line 288 for selective hydrogenation of the unstable compounds MAPD. The MAPD compounds are highly reactive contaminants in a propylene stream. They can be removed by selective hydrogenation in the presence of a catalyst which not only “removes” the contaminants but converts them to valuable product propylene. The stream containing the unconverted MAPD may be called as “Green Oil”. An effluent stream comprising a relatively higher amount of propylene compared to overhead stream comprising C₃₋ hydrocarbons may be withdrawn from the selective hydrogenation unit of the separation zone 290. The effluent stream comprising a relatively higher amount of propylene compared to overhead stream comprising C₃₋ hydrocarbons may be passed to a downstream fractionation column to recover valuable propylene from the effluent stream. The fractionation column (not shown) may also be referred as C₃ splitter. In the fractionation column, an overhead stream comprising propylene may be withdrawn as a product stream from the fractionation column. A bottoms stream comprising propane may be withdrawn. The bottoms stream from the fractionation column may be recycled or recovered for further use. The overhead stream comprising propylene may be withdrawn in line 293 from the separation zone 290.

The bottoms stream comprising C₄₊ hydrocarbons from the depropanizer column may be passed to a debutanizer column (not shown). In the debutanizer column, the bottoms stream comprising C₄₊ hydrocarbons may be separated to provide an overhead stream comprising C₄ and lower hydrocarbons. A bottoms stream comprising pyrolysis gasoline or pygas may be withdrawn from the debutanizer column. Some amount of C₄₋ hydrocarbons may remain in the bottoms stream. The bottoms stream comprising pyrolysis gasoline or pygas is withdrawn as pygas stream in line 294 from the separation zone 290. In an exemplary embodiment, the pygas stream in line 294 may be combined with the bottoms stream in line 224 from the quench zone 220 to provide a combined bottoms stream comprising pyrolysis gasoline in line 296. The combined bottoms stream in line 296 may be further passed to BTX extraction.

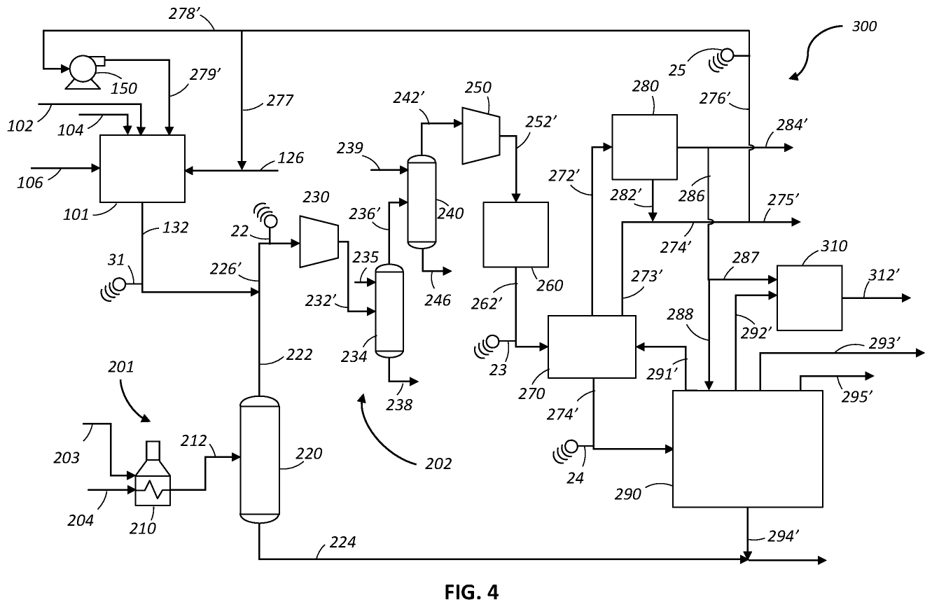

Turning now to FIG. 4, another exemplary embodiment of the integrated process for producing acetylene is addressed with reference to a process and apparatus. Elements of FIG. 4 may have the same configuration as in FIG. 3 and bear the same respective reference number and have similar operating conditions. Elements in FIG. 4 that correspond to elements in FIG. 3 but have a different configuration bear the same reference numeral as in FIG. 3 but are marked with a prime symbol (′).

As shown in FIG. 4, the reactor effluent stream in line 132 from the pyrolytic reactor 101 may be passed in its entirety to the product recovery unit 202 of the steam cracking unit 201. Accordingly, the pyrolytic reactor 101 may be integrated with the steam cracking process via the reactor effluent stream in line 132. The integrated process as shown in FIG. 4, omits the requirement of separation zone downstream of the reaction zone 130 of the pyrolytic reactor 101. Instead, the scheme as shown in FIG. 4 utilizes the acetylene processing unit 310 of the product recovery unit 202 of the steam cracking process to separate and recover acetylene from the reactor effluent stream in line 132 which is produced in the pyrolytic reactor 101.

The cooled reactor effluent stream comprising the reaction mixture in line 132 may be passed to the product recovery unit 202 of the steam cracking process. Accordingly, the pyrolytic reactor may be integrated with the steam cracking process via the reactor effluent stream comprising the reaction mixture in line 132. The reactor effluent stream comprising the reaction mixture in line 132 may be separated in the integrated product recovery unit 202 to provide an acetylene stream and a fuel gas stream comprising methane, carbon oxides and the hydrogen.

As shown, the reactor effluent stream comprising the reaction mixture in line 132 may be combined with the cracked gas effluent stream in the overhead line 222 to provide a combined overhead stream in line 226′. The combined overhead stream in line 226′ may be separated into the product recovery unit 202 to recover hydrocarbons present therein and provide the gas stream comprising methane. The combined overhead stream in line 226′ may be passed to the first stage compressor 230. Alternately, reactor effluent stream comprising the reaction mixture in line 132 and the cracked gas effluent stream in the overhead line 222 may be passed separately to the first stage compressor 230 and compressed therein. A compressed effluent stream in line 232′ is obtained from the first stage compressor 230. The compressed effluent stream in line 232′ may be passed through the trayed or packed absorption column 234 where it is scrubbed by means of an absorbent liquid such as an aqueous solution fed by line 235 to remove acid gases including hydrogen sulfide and carbon dioxide by absorbing them into the aqueous solution. However, if the pressure of the pyrolytic reactor effluent stream is boosted to be at least equal to the packed absorption column 234 inlet pressure, the reactor effluent stream comprising the reaction mixture in line 132 may be passed to the packed absorption column 234. Accordingly, a pump can used to compress the reactor effluent stream in line 132 and a compressed reactor effluent stream can be passed to the packed absorption column 234.

A resultant effluent stream can be taken out from the overhead outlet of the packed absorption column 234 in an overhead line 236′. The resultant effluent stream in the overhead line 236′ may be further cleaned by directing the resultant effluent stream in the overhead line 236′ to the caustic scrubber 240 for removal of remnant entrained acid gases such as CO₂. A sweetened stream is obtained from the caustic scrubber 240 in an overhead line 242′. The sweetened stream in the overhead line 242′ may be compressed in the second stage compressor 250. A compressed sweetened stream in line 252′ is withdrawn from the second stage compressor 250. The compressed sweetened stream in line 252′ may be directed to the drying unit 260. A dry stream in line 262′ may be directed to the downstream recovery unit for removal of the acetylene and remaining components contained therein. The dry stream in line 262′ may be passed to the cold box 270 to separate the fuel gas stream from the dry stream in line 262.

The cold box 270 separates the dry stream in line 262′ into a liquid stream in line 274′ comprising C₂₊ hydrocarbons and the fuel gas stream. Accordingly, a light gas stream in line 272′ comprising hydrogen is produced in the cold box 270 which may be further processes to recover hydrogen. Also produced in the cold box 270 as a result of associated multiple cooling and flashing steps is a light recycle stream 273′ comprising hydrogen and methane. The liquid stream in line 274′ comprising C₂₊ hydrocarbons and the light gas stream in line 272′ comprising hydrogen and methane are withdrawn from the cold box 270. The light gas stream in line 272′ may be passed to pressure swing adsorption (PSA) unit 280 for recovery of hydrogen.

The liquid stream in line 274′ comprising C₂₊ hydrocarbons from the cold box 270 may be directed to the separation zone 290 for removal acetylene and other valuable components present therein. The liquid stream in line 274 may be first passed to the demethanizer column (not shown) to separate methane from the liquid stream in line 274′. Methane may be recovered in the overhead stream. The overhead stream comprising the recovered methane in line 291′ may be passed to the cold box 270. Alternatively, the overhead stream comprising methane in line 291′ may be combined with the fuel gas stream in line 274′. A bottoms stream comprising remnant hydrocarbons may be withdrawn from the demethanizer column.

The bottoms stream withdrawn from the demethanizer column may be passed to a deethanizer column (not shown). An overhead stream comprising C₂₋ hydrocarbons and a bottoms stream comprising C₃₊ hydrocarbons may be withdrawn from the deethanizer column. Some C₂₋ hydrocarbons may also remain present in the bottoms stream of the deethanizer column. The overhead stream comprising C₂₋ hydrocarbons may be withdrawn in line 292′. The acetylene produced in the pyrolytic reactor 101 remains in the overhead stream comprising C₂₋ hydrocarbons may be withdrawn in line 292′ which can be separated. Accordingly, the overhead stream comprising C₂₋ hydrocarbons in line 292′ may be passed to the acetylene processing unit 310. The acetylene processing unit 310 may be used to separate acetylene from the overhead stream comprising C₂₋ hydrocarbons in line 292′. Alternatively, the acetylene processing unit 310 can be used to convert acetylene to more valuable hydrocarbons including but not limited to ethylene. Rest of the process is the same as described herein above for FIG. 3.

The integrated process as shown in FIG. 4 utilizes the separation zone 290 of the product recovery unit 202 of the steam cracking process to separate acetylene produced in the pyrolytic reactor 101 and other valuable hydrocarbons produced in the integrated process. The current scheme avoids the use of a dedicated separation zone for the pyrolytic reactor for separating acetylene from the reactor effluent stream in line 132. Also, the gas stream comprising methane in line 276 recovered from the product recovery unit 202 can be injected into the pyrolytic reactor 101 as a fuel or feed or both.

Any of the above lines, conduits, units, devices, vessels, surrounding environments, zones or similar may be equipped with one or more monitoring components including sensors, measurement devices, data capture devices or data transmission devices. Signals, process or status measurements, and data from monitoring components may be used to monitor conditions in, around, and on process equipment. Signals, measurements, and/or data generated or recorded by monitoring components may be collected, processed, and/or transmitted through one or more networks or connections that may be private or public, general or specific, direct or indirect, wired or wireless, encrypted or not encrypted, and/or combination(s) thereof; the specification is not intended to be limiting in this respect. Further, the figures show one or more exemplary sensors such as 21, 22, 23, 24, 25, and 31 located on one or more conduits. Nevertheless, there may be sensors present on every stream so that the corresponding parameter(s) can be controlled accordingly.

Signals, measurements, and/or data generated or recorded by monitoring components may be transmitted to one or more computing devices or systems. Computing devices or systems may include at least one processor and memory storing computer-readable instructions that, when executed by the at least one processor, cause the one or more computing devices to perform a process that may include one or more steps. For example, the one or more computing devices may be configured to receive, from one or more monitoring component, data related to at least one piece of equipment associated with the process. The one or more computing devices or systems may be configured to analyze the data. Based on analyzing the data, the one or more computing devices or systems may be configured to determine one or more recommended adjustments to one or more parameters of one or more processes described herein. The one or more computing devices or systems may be configured to transmit encrypted or unencrypted data that includes the one or more recommended adjustments to the one or more parameters of the one or more processes described herein.

### SPECIFIC EMBODIMENTS

While the following is described in conjunction with specific embodiments, it will be understood that this description is intended to illustrate and not limit the scope of the preceding description and the appended claims.

A first embodiment of the present disclosure is an integrated process for producing acetylene, comprising recovering a fuel gas stream from a product recovery unit; separating a gas stream comprising methane from the fuel gas stream in the product recovery unit; combusting a fuel and an oxidizer in a combustion zone of a pyrolytic reactor to create a combustion gas stream, wherein the pyrolytic reactor is integrated with the product recovery unit via the gas stream comprising methane; accelerating a velocity of the combustion gas stream from subsonic to supersonic in an expansion zone of the pyrolytic reactor to provide a supersonic combustion gas stream; injecting a light hydrocarbon stream comprising all or a first portion of the gas stream comprising methane into the supersonic combustion gas stream to create a mixed stream including the light hydrocarbon stream; transitioning the velocity of the mixed stream from supersonic to subsonic in a reaction zone of the pyrolytic reactor to produce a reaction mixture comprising acetylene, methane, carbon oxides, and hydrogen; and separating the reaction mixture to provide an acetylene stream. An embodiment of the present disclosure is one, any or all of prior embodiments in this paragraph up through the first embodiment in this paragraph, wherein separating the reaction mixture comprises passing the reaction mixture to a separation zone of the pyrolytic reactor to separate the reaction mixture into the acetylene stream and a byproduct stream comprising methane, carbon oxides and hydrogen. An embodiment of the present disclosure is one, any or all of prior embodiments in this paragraph up through the first embodiment in this paragraph, wherein the acetylene is absorbed in solvent in an absorber in the separation zone to recover the acetylene stream. An embodiment of the present disclosure is one, any or all of prior embodiments in this paragraph up through the first embodiment in this paragraph further that comprises separating the reaction mixture in an integrated product recovery unit to provide the acetylene stream and the fuel gas stream. An embodiment of the present disclosure is one, any or all of prior embodiments in this paragraph up through the first embodiment in this paragraph, wherein the first portion ranges from 0 to 100 vol % of the gas stream comprising methane. An embodiment of the present disclosure is one, any or all of prior embodiments in this paragraph up through the first embodiment in this paragraph further comprises injecting a second portion of the gas stream comprising methane into the combustion zone of the pyrolytic reactor. An embodiment of the present disclosure is one, any or all of prior embodiments in this paragraph up through the first embodiment in this paragraph, wherein the second portion ranges from 0 to 100 vol % of the gas stream comprising methane. An embodiment of the present disclosure is one, any or all of prior embodiments in this paragraph up through the first embodiment in this paragraph further comprises compressing the second portion of the gas stream comprising methane to obtain a compressed gas stream and then injecting the compressed gas stream into the supersonic combustion gas stream. An embodiment of the present disclosure is one, any or all of prior embodiments in this paragraph up through the first embodiment in this paragraph, wherein the product recovery unit integrated with the pyrolytic reactor is a product recovery unit of a steam cracking process. An embodiment of the present disclosure is one, any or all of prior embodiments in this paragraph up through the first embodiment in this paragraph, wherein recovering the fuel gas stream comprises passing a hydrocarbonaceous feedstock to a cracking zone of the steam cracking process to pyrolyze the hydrocarbonaceous feedstock in the presence of steam to provide a steam cracked effluent stream; separating the steam cracked effluent stream into a cracked gas effluent stream comprising C2-C4 olefins, methane, carbon oxides, and hydrogen and a liquid stream; separating the cracked gas effluent stream in the product recovery unit of the steam cracking process to provide the fuel gas stream; and separating and recovering the gas stream comprising methane in the product recovery unit of the steam cracking process from the fuel gas stream. An embodiment of the present disclosure is one, any or all of prior embodiments in this paragraph up through the first embodiment in this paragraph, wherein the hydrocarbonaceous feedstock is selected from one or more of naphtha, kerosene, condensate, atmospheric gas oil, vacuum gas oil, hydrocrackate, and crude oil. An embodiment of the present disclosure is one, any or all of prior embodiments in this paragraph up through the first embodiment in this paragraph further comprising combining the vapor stream with the byproduct stream to provide a combined vapor stream and separating the combined vapor stream in the product recovery unit to provide the gas stream comprising methane.

A second embodiment of the present disclosure is an integrated process for producing acetylene, comprising combusting a fuel and an oxidizer in a combustion zone of a pyrolytic reactor to create a combustion gas stream; accelerating a velocity of the combustion gas stream from subsonic to supersonic in an expansion zone of the pyrolytic reactor; injecting a light hydrocarbon stream into the supersonic combustion gas stream to create a mixed stream comprising the light hydrocarbon; transitioning the velocity of the mixed stream from supersonic to subsonic in a reaction zone of the pyrolytic reactor to produce a reaction mixture comprising acetylene, methane, carbon oxides, and hydrogen; passing the reaction mixture to a product recovery unit integrated with the pyrolytic reactor; and separating the reaction mixture in the integrated product recovery unit to provide an acetylene stream and a fuel gas stream comprising methane, carbon oxides and the hydrogen. An embodiment of the present disclosure is one, any or all of prior embodiments in this paragraph up through the second embodiment in this paragraph further comprising recovering a gas stream comprising methane from the fuel gas stream in the integrated product recovery unit; and injecting all or a first portion of the gas stream comprising methane into the supersonic combustion gas stream to create the mixed stream. An embodiment of the present disclosure is one, any or all of prior embodiments in this paragraph up through the second embodiment in this paragraph, wherein the first portion ranges from 0 to 100 vol % of the gas stream comprising methane. An embodiment of the present disclosure is one, any or all of prior embodiments in this paragraph up through the second embodiment in this paragraph that further comprises injecting a second portion of the gas stream comprising methane into the combustion zone. An embodiment of the present disclosure is one, any or all of prior embodiments in this paragraph up through the second embodiment in this paragraph, wherein the second portion ranges from 0 to 100 vol % of the gas stream comprising methane. An embodiment of the present disclosure is one, any or all of prior embodiments in this paragraph up through the second embodiment in this paragraph, wherein the product recovery unit is a product recovery unit of a steam cracking process. An embodiment of the present disclosure is one, any or all of prior embodiments in this paragraph up through the second embodiment in this paragraph, wherein separating the reaction mixture in the integrated product recovery unit comprises passing a hydrocarbonaceous feedstock to a cracking zone of the steam cracking process, wherein the hydrocarbonaceous feedstock is pyrolyzed in the presence of steam to provide a steam cracked effluent stream; separating the steam cracked effluent stream into a cracked gas effluent stream comprising C2-C4 olefins, methane, carbon oxides, and hydrogen and a liquid stream; combining and compressing the reaction mixture and the cracked gas effluent stream to provide a compressed stream; separating the compressed stream in the product recovery unit of the steam cracking process to provide the fuel gas stream and the acetylene stream; and separating/recovering the gas stream comprising methane in the product recovery unit from the fuel gas stream.

A third embodiment of the present disclosure is an integrated process for producing acetylene, comprising combusting a fuel and an oxidizer in a combustion zone of a pyrolytic reactor to create a combustion gas stream; accelerating a velocity of the combustion gas stream from subsonic to supersonic in an expansion zone of the pyrolytic reactor; injecting a light hydrocarbon stream into the supersonic combustion gas stream to create a mixed stream including the light hydrocarbon; transitioning the velocity of the mixed stream from supersonic to subsonic in a reaction zone of the pyrolytic reactor to produce a reaction mixture comprising acetylene, methane, carbon oxides, and hydrogen; separating the reaction mixture in a separation zone of the pyrolytic reactor into an acetylene stream and a byproduct stream comprising the methane, carbon oxides and the hydrogen; passing the byproduct stream to a fuel gas recovery unit integrated with the pyrolytic reactor, wherein the pyrolytic reactor is integrated with the fuel gas recovery unit via the byproduct stream; separating the byproduct stream in the fuel gas recovery unit to provide a gas stream comprising methane; and injecting all or a first portion of the gas stream comprising methane into the supersonic combustion gas stream.

Without further elaboration, it is believed that using the preceding description that one skilled in the art can utilize the present disclosure to its fullest extent and easily ascertain the essential characteristics of this disclosure, without departing from the spirit and scope thereof, to make various changes and modifications of the present disclosure and to adapt it to various usages and conditions. The preceding preferred specific embodiments are, therefore, to be construed as merely illustrative, and not limiting the remainder of the disclosure in any way whatsoever, and that it is intended to cover various modifications and equivalent arrangements included within the scope of the appended claims.

In the foregoing, all temperatures are set forth in degrees Celsius and, all parts and percentages are by weight, unless otherwise indicated.

1. An integrated process for producing acetylene, comprising:
recovering a fuel gas stream from a product recovery unit;
separating a gas stream comprising methane from the fuel gas stream in the product recovery unit;
combusting a fuel and an oxidizer in a combustion zone of a pyrolytic reactor to create a combustion gas stream, wherein the pyrolytic reactor is integrated with the product recovery unit via the gas stream comprising methane;
accelerating a velocity of the combustion gas stream from subsonic to supersonic in an expansion zone of the pyrolytic reactor to provide a supersonic combustion gas stream;
injecting a light hydrocarbon stream comprising all or a first portion of the gas stream comprising methane into the supersonic combustion gas stream to create a mixed stream including the light hydrocarbon stream;
transitioning the velocity of the mixed stream from supersonic to subsonic in a reaction zone of the pyrolytic reactor to produce a reaction mixture comprising acetylene, methane, carbon oxides, and hydrogen; and
separating the reaction mixture to provide an acetylene stream.

2. The process of claim 1, wherein separating the reaction mixture comprises passing the reaction mixture to a separation zone of the pyrolytic reactor to separate the reaction mixture into the acetylene stream and a byproduct stream comprising methane, carbon oxides and hydrogen.

3. The process of claim 2, wherein the acetylene is absorbed in solvent in an absorber in the separation zone to recover the acetylene stream.

4. The process of claim 1 further that comprises separating the reaction mixture in an integrated product recovery unit to provide the acetylene stream and the fuel gas stream.

5. The process of claim 1, wherein the first portion ranges from 0 to 100 vol % of the gas stream comprising methane.

6. The process of claim 1 further comprises injecting a second portion of the gas stream comprising methane into the combustion zone of the pyrolytic reactor.

7. The process of claim 6, wherein the second portion ranges from 0 to 100 vol % of the gas stream comprising methane.

8. The process of claim 6 further comprises compressing the second portion of the gas stream comprising methane to obtain a compressed gas stream and then injecting the compressed gas stream into the supersonic combustion gas stream.

9. The process of claim 1, wherein the product recovery unit integrated with the pyrolytic reactor is a product recovery unit of a steam cracking process.

10. The process of claim 9, wherein recovering the fuel gas stream comprises:
passing a hydrocarbonaceous feedstock to a cracking zone of the steam cracking process to pyrolyze the hydrocarbonaceous feedstock in the presence of steam to provide a steam cracked effluent stream;
separating the steam cracked effluent stream into a cracked gas effluent stream comprising C2-C4 olefins, methane, carbon oxides, and hydrogen and a liquid stream;
separating the cracked gas effluent stream in the product recovery unit of the steam cracking process to provide the fuel gas stream; and
separating and recovering the gas stream comprising methane in the product recovery unit of the steam cracking process from the fuel gas stream.

11. The process of claim 10, wherein the hydrocarbonaceous feedstock is selected from one or more of naphtha, kerosene, condensate, atmospheric gas oil, vacuum gas oil, hydrocrackate, and crude oil.

12. The process of claim 10 further comprising combining the vapor stream with the byproduct stream to provide a combined vapor stream and separating the combined vapor stream in the product recovery unit to provide the gas stream comprising methane.

13. An integrated process for producing acetylene, comprising
combusting a fuel and an oxidizer in a combustion zone of a pyrolytic reactor to create a combustion gas stream;
accelerating a velocity of the combustion gas stream from subsonic to supersonic in an expansion zone of the pyrolytic reactor;
injecting a light hydrocarbon stream into the supersonic combustion gas stream to create a mixed stream comprising the light hydrocarbon;
transitioning the velocity of the mixed stream from supersonic to subsonic in a reaction zone of the pyrolytic reactor to produce a reaction mixture comprising acetylene, methane, carbon oxides, and hydrogen;
passing the reaction mixture to a product recovery unit integrated with the pyrolytic reactor; and
separating the reaction mixture in the integrated product recovery unit to provide an acetylene stream and a fuel gas stream comprising methane, carbon oxides and the hydrogen.

14. The process of claim 13 further comprising:
recovering a gas stream comprising methane from the fuel gas stream in the integrated product recovery unit; and
injecting all or a first portion of the gas stream comprising methane into the supersonic combustion gas stream to create the mixed stream.

15. The process of claim 14, wherein the first portion ranges from 0 to 100 vol % of the gas stream comprising methane.

16. The process of claim 14 that further comprises injecting a second portion of the gas stream comprising methane into the combustion zone.

17. The process of claim 16, wherein the second portion ranges from 0 to 100 vol % of the gas stream comprising methane.

18. The process of claim 13, wherein the product recovery unit is a fuel gas recovery unit of a steam cracking process.

19. The process of claim 18, wherein separating the reaction mixture in the integrated product recovery unit comprises:
passing a hydrocarbonaceous feedstock to a cracking zone of the steam cracking process, wherein the hydrocarbonaceous feedstock is pyrolyzed in the presence of steam to provide a steam cracked effluent stream;
separating the steam cracked effluent stream into a cracked gas effluent stream comprising C2-C4 olefins, methane, carbon oxides, and hydrogen and a liquid stream;
combining and compressing the reaction mixture and the cracked gas effluent stream to provide a compressed stream;
separating the compressed stream in the product recovery unit of the steam cracking process to provide the fuel gas stream and the acetylene stream; and
separating/recovering the gas stream comprising methane in the product recovery unit from the fuel gas stream.

20. An integrated process for producing acetylene, comprising
combusting a fuel and an oxidizer in a combustion zone of a pyrolytic reactor to create a combustion gas stream;
accelerating a velocity of the combustion gas stream from subsonic to supersonic in an expansion zone of the pyrolytic reactor;
injecting a light hydrocarbon stream into the supersonic combustion gas stream to create a mixed stream including the light hydrocarbon;
transitioning the velocity of the mixed stream from supersonic to subsonic in a reaction zone of the pyrolytic reactor to produce a reaction mixture comprising acetylene, methane, carbon oxides, and hydrogen;
separating the reaction mixture in a separation zone of the pyrolytic reactor into an acetylene stream and a byproduct stream comprising the methane, carbon oxides and the hydrogen;
passing the byproduct stream to a product recovery unit integrated with the pyrolytic reactor, wherein the pyrolytic reactor is integrated with the product recovery unit via the byproduct stream;
separating the byproduct stream in the product recovery unit to provide a gas stream comprising methane; and
injecting all or a first portion of the gas stream comprising methane into the supersonic combustion gas stream.
